(19) United States
(12) Patent Application Publication
(10) Pub. No.: US 2008/0286818 A1
(43) Pub. Date: Nov. 20, 2008

(54) BLOOD SAMPLE HANDLING METHODS FOR IMPROVED ASSAYS FOR MYELOPEROXIDASE

(76) Inventors: Saul A. Datwyler, Evanston, IL (US); Stephen C. Hsu, Buffalo Grove, IL (US); Matthew S. Matias, Green Oaks, IL (US); David P. Pacenti, Gurnee, IL (US); Jessie W. Shih, Lake Forest, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/750,507

(22) Filed: May 18, 2007

(51) Int. Cl. G01N 33/573; C12Q 1/28

(52) U.S. Cl. 435/7.93; 435/28; 435/7.92; 435/7.94

(57) ABSTRACT

The invention provides a method for determining concentration of myeloperoxidase (MPO) in a human blood sample comprising: (a) providing a human peripheral blood sample stored under MPO preservation conditions; and (b) determining concentration of MPO in a plasma sample derived from the human peripheral blood sample. In a preferred embodiment, the MPO preservation conditions used in the invention comprise storage of the human peripheral blood sample in a plasma collection tube containing a leukocyte MPO secretion inhibitor. In this embodiment, the leukocyte MPO secretion inhibitor is preferably ethylene diamine tetraacetic acid (EDTA). The assays used in the inventive methods can comprise any clinically useful assay for determining MPO plasma concentration, including sandwich and competitive immunoassays, clinical chemistry assays and enzymatic assays.

### FIELD OF THE INVENTION

This invention relates to diagnostic assays to determine the concentration levels of myeloperoxidase (MPO) in a patient blood sample, and in particular relates to use of improved blood sample handling methods to preserve original MPO levels in the sample.

### BACKGROUND OF THE INVENTION

Plasma myeloperoxidase levels determined in patient blood samples collected during patient assessment in the Emergency Room for acute coronary syndrome (ACS) have been shown predictive of the risk for major adverse cardiac events (MACE), such as myocardial infarction, need for revascularization or death, over 30 day and six month periods following the sample collection, see M. Brennan et al., N. Eng. J. Med. (2003) 349(17): 1595-1604. Determination of MPO levels in suspected ACS patient blood samples by assays are thus of clinical interest for managing ACS patients.

Sample collection tube type and specimen handling can affect the measured amount of an analyte in many types of assays commonly used in clinical laboratories to assay blood samples, including sandwich and competitive immunoassays, clinical chemistry assays and enzymatic assays. In particular, MPO is known to be present in leukocytes as well as free in the plasma. Leukocyte MPO can be released into the plasma depending on sample handling and specimen tube used to collect the sample. Release of MPO from leukocytes into the plasma fraction due to inadequate sample handling can lead to erroneous determination of MPO plasma levels. This erroneous determination of higher MPO plasma levels can lead to inaccurate stratification of ACS patients, and consequently wrong treatment selection.

However, current pre-analytical patient blood sample handling does not involve any specific steps aimed at preservation of original MPO levels. Rather, current MPO assays follow the blood storage procedures used with troponin measurements, namely collecting samples as lithium heparin plasma or serum. For example, the United States Food and Drug Administration has cleared one MPO assay for clinical use, the CardioMPO™ (a trademark of PrognostiX, Inc. (Cleveland, Ohio)) Enzyme Immunoassay Reagent Kit marketed by PrognostiX. The CardioMPO assay is an enzyme-linked immunosorbent assay (ELISA). The CardioMPO package insert states that the patient blood sample “should be stored in lithium heparin collection tubes”, and that the tubes should be placed on ice or at 2 to 8 degrees centigrade immediately and then stored at 2 to 8 degrees centigrade until processed, CardioMPO package insert at page 7.

The current blood sample handling procedures for MPO do not take into account the reality that most blood samples will be exposed to at least some time at room temperature storage. The room temperature storage may also be for an extended time. Exposure to room temperature conditions can occur at any point before the analysis. Transportation of the sample to the laboratory from the location of blood draw (such as Emergency Department or Intensive Care Unit) is typically done at room temperature and once in the lab there can be waiting time for centrifugation. The automated blood analyzers in clinical laboratories generally do not use cold storage conditions for the samples, so the blood samples are exposed to room temperature conditions during processing. In addition, clinical laboratories in the US have begun implementing automated systems with multiple analyzer stations linked by automated conveyor systems. These automated systems also increase the likelihood a blood sample will be exposed to room temperature storage for extended times. Last, in addition to the reality of likely exposure to room temperature conditions, current practice does not involve any tracking or monitoring of the actual exposure of a sample to be tested for MPO levels to room temperature.

Applicants have determined that pre-analytical sample handling methods currently used in advance of MPO assay allows MPO to leak out of the leukocytes during clotting of serum and preparation of lithium heparin plasma, which causes an elevation in MPO levels. It is therefore an object of the invention to provide for use with MPO assays methods for patient blood sample handling specifically intended to preserve original patient MPO levels for accurate assay. The inventive methods advantageously improve MPO determinations in any clinical assay by preserving original MPO levels.

### SUMMARY OF THE INVENTION

The invention provides a method for determining concentration of myeloperoxidase (MPO) in a human blood sample comprising: (a) providing a human peripheral blood sample stored under MPO preservation conditions; and (b) determining concentration of MPO in a plasma sample derived from the human peripheral blood sample. In a preferred embodiment, the MPO preservation conditions used in the invention comprise storage of the human peripheral blood sample in a plasma sample collection tube containing a leukocyte MPO secretion inhibitor, which is selected to inhibit secretion of MPO by leukocytes as occurs at room temperature. In this embodiment, the leukocyte MPO secretion inhibitor is preferably ethylene diamine tetraacetic acid (EDTA). The assays used in the inventive methods can comprise any clinically useful assay for determining MPO plasma concentration, including sandwich and competitive immunoassays, clinical chemistry assays and enzymatic assays. Preferably the MPO measurement is done using an immunoassay, and more preferably, a sandwich immunoassay. The invention is particularly useful for MPO assays run on automated analyzer systems, where samples for MPO testing are often exposed to room temperature conditions. Through use of the invention, MPO levels can be preserved to essentially original collection levels during exposure to room temperature conditions for at least 8 hours, and potentially up to 24 hours prior to centrifugation and separation of the plasma.

In a second embodiment, the invention comprises a method for determining the concentration of myeloperoxidase (MPO) in a human blood sample comprising: (a) providing a human peripheral blood sample stored under MPO preservation conditions; and (b) determining concentration of MPO in a plasma sample derived from the human peripheral blood sample. In a preferred embodiment, the MPO preservation conditions used in the invention comprise storage of the human peripheral blood sample in a plasma sample collection tube containing a leukocyte MPO secretion inhibitor, which is selected to inhibit secretion of MPO by leukocytes as occurs at 2-8 degrees Celsius. In this embodiment, the leukocyte MPO secretion inhibitor is preferably ethylene diamine tetraacetic acid (EDTA) or sodium citrate and the assays used in the inventive methods can comprise any clinically useful assay for determining MPO plasma concentration, including sandwich and competitive immunoassays, clinical chemistry assays and enzymatic assays. Preferably the MPO measurement is done using an immunoassay, and more preferably, a sandwich immunoassay. Through use of this embodiment, MPO levels can be preserved to essentially original collection levels during exposure to 2-8 degrees Celsius for up to 8 hours prior to centrifugation and separation of the plasma fraction.

In another embodiment, the invention comprises a method for determining concentration of myeloperoxidase (MPO) in a human blood sample comprising: (a) providing a human peripheral blood sample stored under MPO preservation conditions; (b) checking the MPO preservation conditions status of the human peripheral blood sample; and (c) determining concentration of MPO in a plasma sample derived from the human peripheral blood sample. This embodiment is particularly advantageous for use with an automated analytical instrument, such as an automated immunoassay analyzer. The analyzer can be programmed to check on the MPO preservation conditions used for the sample, and where unacceptable preservation conditions, such as storage at room temperature, are determined as present, the analyzer can send an error message that it is unable to perform the MPO assay, because of the likelihood of inaccurately high MPO results.

The invention has significant capability to provide improved determination of blood concentration levels of MPO. The inventive methods enable more accurate preservation of and measurement of MPO levels and consequently more accurate patient stratification and treatment selection.

### DETAILED DESCRIPTION OF THE INVENTION

As used herein, the term “blood sample” refers to a human whole blood sample or a plasma fraction derived therefrom. Preferably, the blood sample is a human peripheral blood sample or a plasma fraction derived therefrom.

As used herein, the terms “antibody” and “antibodies” refer to monoclonal antibodies, multispecific antibodies, human antibodies, humanized antibodies (fully or partially humanized), animal antibodies (such as, but not limited to, a bird (for example, a duck or goose), a shark or whale, a mammal, including a non-primate (for example, a cow, pig, camel, llama, horse, goat, rabbit, sheep, hamsters, guinea pig, cat, dog, rat, mouse, etc) or a non-human primate (for example, a monkey, such as a cynomologous monkey, a chimpanzee, etc), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies (including, for example, anti-Id antibodies to antibodies of the present invention), and functionally active epitope-binding fragments of any of the above. In particular, antibodies include immunoglobulin molecules and immunologically active fragments of immunoglobulin molecules, namely, molecules that contain an antigen binding site. Immunoglobulin molecules can be of any type (for example, IgG, IgE, IgM, IgD, IgA and IgY), class (for example, IgG₁, IgG₂, IgG₃, IgG₄, IgA₁and IgA₂) or subclass. An antibody whose affinity (namely, K_{D}, k_{d} or kₐ) has been increased or improved via the screening of a combinatory antibody library that has been prepared using bio-display, is referred to herein as an “affinity maturated antibody”.

As used herein, the term “sample collection tube” or “sample tube” refers to any type of container used to collect and store a human blood sample for shipment to an analytical processing site. Preferably, the sample tube is made of a suitable plastic material that is non-reactive with the stabilizing agents and does not interfere with the biological sample. Suitable plastic materials are known in the art. Preferred plastic material include any type of polyethylene terepththlate (PET) or polypropylene. Sample tubes are generally made by a suitable injection molding process as known in the art. The sample tube can be of any design, including nested designs, such as described in U.S. Pat. No. 6,910,597, M. Iskra, “Collection Container Assembly”. Preferred sample tubes for use in the invention are commercially available from Becton Dickinson (Franklin Lakes, N.J.).

The invention provides a method for determining concentration of myeloperoxidase (MPO) in a human blood sample comprising: (a) providing a human peripheral blood sample stored under MPO preservation conditions; and (b) determining concentration of MPO in a plasma sample derived from the human peripheral blood sample. In a preferred embodiment, the MPO preservation conditions used in the invention comprise storage of the human peripheral blood sample in a plasma sample tube containing a leukocyte MPO secretion inhibitor. In this embodiment, the leukocyte MPO secretion inhibitor is preferably ethylene diamine tetraacetic acid (EDTA).

Preservation of original plasma MPO levels from collection time until measurement is the main objective of the invention, and Applicants determined that this objective can be met by the use of the leukocyte MPO secretion inhibitor. In this regard, a leukocyte MPO secretion inhibitor can be any reagent added to the blood sample on or shortly after collection into the sample tube, that inhibits release of MPO from leukocytes present in the sample, particularly release at room temperature. The inhibition of MPO release by the leukocytes (by the inhibitor) occurs prior to the processing, preferably by centrifugation methods, of the whole blood sample into the plasma fraction, the buffy coat interface next to the plasma fraction (where the leukocytes are) and the red blood cells. Preferred leukocyte MPO secretion inhibitors include any salt of EDTA, including sodium or potassium salts. Preferred EDTA salts include dipotassium and tripotassium salts. The leukocyte MPO secretion inhibitor is included in any suitable concentration, and amounts used in commercially available sample tubes for whole blood are acceptable.

In a second embodiment, after collection into a sample tube with a leukocyte MPO secretion inhibitor, it is preferable that the sample tube be immediately placed on ice or stored at a temperature in a range of 2 to 8 degrees Celsius, until processed for MPO determination. Although it is possible to use a lower range than 2 to 8 degrees, this is not preferred and should be avoided to lessen the likelihood the sample will freeze. In this embodiment, the leukocyte MPO secretion inhibitor includes any salt of EDTA, including sodium or potassium salts. Preferred EDTA salts include dipotassium and tripotassium salts. Other suitable leukocyte MPO secretion inhibitors in this embodiment include any citrate salt, preferably sodium citrate.

In another embodiment, the invention involves an intermediate step before the MPO assay is performed. This embodiment comprises a method for determining concentration of myeloperoxidase (MPO) in a human blood sample comprising: (a) providing a human peripheral blood sample stored under MPO preservation conditions; (b) checking the MPO preservation conditions status of the human peripheral blood sample; and (c) determining concentration of MPO in a plasma sample derived from the human peripheral blood sample. This embodiment is particularly advantageous for use with an automated analytical instrument, such as an automated immunoassay analyzer. The analyzer can be programmed to check on the MPO preservation conditions used for the sample, and where potentially unacceptable preservation conditions, such as storage at room temperature for over 8 hours before sample centrifugation and plasma separation, are determined as present, the analyzer can be programmed to send an error message that it is unable to perform the MPO assay, because of the likelihood of inaccurately high results. In this embodiment, the analyzer would proceed to process a sample stored under MPO preservation conditions, where the room temperature storage is less than 8 hours.

The assays used in the inventive methods can comprise any clinically useful assay for determining MPO plasma concentration, including sandwich and competitive immunoassays, clinical chemistry assays and enzymatic assays. The immunoassays of the invention can be conducted using any format known in the art, such as, but not limited to, a sandwich format, a competitive inhibition format (including both forward or reverse competitive inhibition assays) or in a fluorescence polarization format. Preferably the MPO measurement is done using an immunoassay, and more preferably, a sandwich immunoassay.

In immunoassays for the qualitative detection of an analyte in a test sample, at least one antibody or protein that binds to the analyte is contacted with at least one blood sample or blood plasma fraction. Any suitable antibodies or binding proteins that bind to MPO can be used in the inventive immunoassays. Antibodies to MPO are known in the art, and any of these can be used. It is preferred to use the two monoclonal antibodies that are described in Example 1 below. Because MPO is present as a dimer, it is also possible to use a single antibody as the pair of antibodies in a sandwich assay, ie as the capture antibody and the conjugate antibody.

The antibody-MPO immune complexes can then be detected using any suitable technique. For example, the antibody can be labeled with a detectable label to detect the presence of the antibody-MPO complex. Any suitable label can be used. The selection of a particular label is not critical, but the chosen label must be capable of producing a detectable signal either by itself or in conjunction with one or more additional substances.

Useful detectable labels, their attachment to antibodies or to other binding proteins and detection techniques therefore are known in the art. Any detectable label known in the art can be used. For example, the detectable label can be a radioactive label, such as, ³H, ¹²⁵I, ³⁵S, ¹⁴C, ³²P, ³³P, an enzymatic label, such as horseradish peroxidase, alkaline peroxidase, glucose 6-phosphate dehydrogenase, etc., a chemiluminescent label, such as, acridinium derivatives, luminol, isoluminol, thioesters, sulfonamides, phenanthridinium esters, etc., a fluorescence label, such as, fluorescein (5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, etc.), rhodamine, phycobiliproteins, R-phycoerythrin, quantum dots (zinc sulfide-capped cadmium selenide), a thermometric label or an immuno-polymerase chain reaction label. An introduction to labels, labeling procedures and detection of labels is found in Polak and Van Noorden, Introduction to Immunocytochemistry, 2^{nd} ed. Springer Verlag, N.Y. (1997) and in Haugland, Handbook of Fluorescent Probes and Research Products, 9^{th} ed., (2002), which is a combined handbook and catalogue published by Molecular Probes, Inc., Eugene, Oreg. each of which is incorporated herein by reference. Preferred labels for use with the invention are chemiluminescent labels such as acridinium-9-carboxamide. Additional detail can be found in Mattingly, P. G., and Adamczyk, M. (2002) Chemiluminescent N-sulfonylacridinium-9-carboxamides and their application in clinical assays, in Luminescence Biotechnology: Instruments and Applications (Dyke, K. V., Ed.) pp 77-105, CRC Press, Boca Raton.

The detectable label can be bound to the analyte or antibody either directly or through a coupling agent. An example of a coupling agent that can be used is EDAC (1-ethyl-3-(3-dimethylaminopropyl)carbodiimide, hydrochloride) that is commercially available from Sigma-Aldrich (St. Louis, Mo.). Other coupling agents that can be used are known in the art. Methods for binding a detectable label to an antibody are known in the art. Additionally, many detectable labels can be purchased or synthesized that already contain end groups that facilitate the coupling of the detectable label to the antibody, such as, N10-(3-sulfopropyl)-N-(3-carboxypropyl)-acridinium-9-carboxamide, otherwise known as CPSP-Acridinium Ester or N10-(3-sulfopropyl)-N-(3-sulfopropyl)-acridinium-9-carboxamide, otherwise known as SPSP-Acridinium Ester.

Alternatively, a second antibody that binds to MPO and that contains a detectable label can be added to the test sample or test sample extract and used to detect the presence of the antibody-MPO complex. Any suitable detectable label can be used in this embodiment.

In using a preferred sandwich type format, at least two antibodies are employed to separate and quantify MPO in the test sample or test sample extract. More specifically, the at least two antibodies bind to different parts of MPO forming an immune complex which is referred to as a “sandwich”. Generally, one or more antibodies can be used to capture the analyte in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, it is preferred that both antibodies binding to the MPO are not diminished by the binding of any other antibody in the assay to its respective binding site. In other words, antibodies should be selected so that the one or more first antibodies brought into contact with a test sample or test sample extract suspected of containing MPO do not bind to all or part of the binding site recognized by the second or subsequent antibodies, thereby interfering with the ability of the one or more second detection antibodies to bind to MPO. In a sandwich assay, the antibodies, preferably, the at least one capture antibody, are used in molar excess amounts of the maximum amount of analyte expected in the test sample or test sample extract. For example, from about 5 μg/mL to about 1 mg/mL of antibody per mL of solid phase containing solution can be used.

In one embodiment, the at least one first capture antibody can be bound to a solid support which facilitates the separation of the first antibody-MPO complex from the test sample. The solid support or “solid phase” used in the inventive immunoassay is not critical and can be selected by one skilled in the art. A solid phase or solid support, as used herein, refers to any material that is insoluble, or can be made insoluble by a subsequent reaction. Useful solid phases or solid supports are known to those in the art and include the walls of wells of a reaction tray, test tubes, polystyrene beads, magnetic beads, nitrocellulose strips, membranes, microparticles such as latex particles, sheep (or other animal) red blood cells, and Duracytes® (a registered trademark of Abbott Laboratories, Abbott Park, Ill.), which are red blood cells “fixed” by pyruvic aldehyde and formaldehyde, and others. Suitable methods for immobilizing peptides on solid phases include ionic, hydrophobic, covalent interactions and the like. The solid phase can be chosen for its intrinsic ability to attract and immobilize the capture reagent. Alternatively, the solid phase can comprise an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid phase and which has the ability to immobilize the capture reagent through a specific binding reaction. The receptor molecule enables the indirect binding of the capture reagent to a solid phase material before the performance of the assay or during the performance of the assay.

Any solid support known in the art can be used, including but not limited to, solid supports made out of polymeric materials in the forms of wells, tubes or beads. The antibody (or antibodies) can be bound to the solid support by adsorption, by covalent bonding using a chemical coupling agent or by other means known in the art, provided that such binding does not interfere with the ability of the antibody to bind the drug. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

It is within the scope of the present invention that the solid phase also can comprise any suitable porous material with sufficient porosity to allow access by detection antibodies and a suitable surface affinity to bind antigens. Microporous structures generally are preferred, but materials with gel structure in the hydrated state may be used as well. Such useful solid supports include but are not limited to nitrocellulose and nylon. It is contemplated that such porous solid supports described herein preferably are in the form of sheets of thickness from about 0.01 to 0.5 mm, preferably about 0.1 mm. The pore size may vary within wide limits, and preferably is from about 0.025 to 15 microns, especially from about 0. 15 to 15 microns. The surface of such supports may be activated by chemical processes which cause covalent linkage of the antigen or antibody to the support. The irreversible binding of the antigen or antibody is obtained, however, in general, by adsorption on the porous material by poorly understood hydrophobic forces.

After the blood sample containing MPO is brought into contact with the at least one first capture antibody, the resulting mixture is incubated to allow for the formation of a first capture antibody (or multiple antibody)-analyte complex. The incubation can be carried out at any suitable pH, including a pH of from about 4.5 to about 10.0, at any suitable temperature, including from about 2° C. to about 45° C., and for a suitable time period from at least about one (1) minute to about eighteen (18) hours, preferably from about 4-20 minutes, most preferably from about 4-6 minutes.

After formation of the labeled complex, the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified detecting the light emitted either visually or by using luminometers, x-ray film, high speed photographic film, a CCD camera, etc. Once the amount of the label in the complex has been quantified, the concentration of analyte in the test sample is determined by use of a standard curve that has been generated using serial dilutions of the analyte of known concentration. Other than using serial dilutions of the analyte, the standard curve can be generated gravimetrically, by mass spectroscopy and by other techniques known in the art.

Any suitable instrumentation or automation can be used in the performance of the determination of MPO levels in the blood sample. It is preferred to carry out the assay in an automated fashion, such as on the Architect® (a registered trademark of Abbott Laboratories, Abbott Park, Ill.) system, which uses chemiluminescence detection of sandwich and competitive immunoassays. The assay can also be carried out in a miniaturized format, such as in a Lab-on-a-Chip device and system, or in a microtiter plate format.

Any suitable control composition for MPO assay can be used in immunoassays implementing the invention. The control compositions generally comprise the MPO antigen to be assayed for along with any desirable additives. A preferred control MPO antigen is available commercially from Athens Research and Technology Inc. (Athens, Ga.).

The following examples illustrate the methods of the invention.

### EXAMPLE 1

### Evaluation of Specimen Collection Tubes

The effect of specimen collection tube anticoagulant or lack of coagulant and glass vs. plastic tube were studied. Blood samples from 15 normal donors collected in 9 different sample tube types were purchased from a commercial vendor. Samples were centrifuged, plasma/serum separated, stored at 2-8 degrees Celsius and shipped to the processing site. The MPO values were measured by assay using an experimental sandwich immunoassay performed on the Architect automated immunoassay instrument (sold by Abbott Laboratories, Abbott Park, Ill.). The experimental Architect assay is described in S. Datwyler et al., “Development of an Automated Myeloperoxidase (MPO) Immunoassay”, Clinical Chemistry. 52. No. 6. Supplement; D-11, 2006. The analytical range of the assay is from 5 to 10,000 pmol/L with precision ranging from 2.4 to 6.0% CV over the range of 250 to 5000 pmol/L. The two monoclonal antibodies used in the experimental Architect assay are 1-1175-509 and 1-2169-715. As used herein, “1-1175-509” (also known as “MPO 1-1175-509”) refers to a monoclonal antibody produced by the murine hybridoma cell line 1-1175-509, which was deposited with the American Type Culture Collection (A.T.C.C) at 10801 University Boulevard, Manassas, Va. 20110 in accordance with the Budapest Treaty on May 16, 2007 and assigned A.T.C.C. Accession No. ______. “1-2169-715” (also known as “MPO 1-2169-715”) refers to a monoclonal antibody produced by the murine hybridoma cell line 1-2169-715, which also was deposited with the A.T.C.C on May 16, 2007 and was assigned A.T.C.C. Accession No. ______.

The sample tube types and measured mean MPO values are in the following Table 1:

TABLE 1

| | | Mean MPO |
|---|---|---|
| Anticoagulant | Type Type | (pmol/L) |
| Lithium Heparin | Plastic | 100.9 |
| PST (Li hep plasma | Plastic | 101.2 |
| separator tube) | | |
| Sodium Heparin | Plastic | 99.5 |
| Lithium Heparin | Glass | 109.6 |
| Serum | Glass | 144.7 |
| Serum | Plastic | 191.6 |
| SST (Serum Separator tube) | Plastic | 149.3 |
| EDTA | Plastic | 91.9 |
| Citrate | Plastic | 93.7 |

The lowest concentration of MPO was found with EDTA and citrate plasma samples. The samples collected in heparin storage tubes showed mean MPO levels approximately 10% higher in value than the preferred EDTA tube. Serum samples were markedly higher in mean MPO level. This data supports that the conventional use of lithium heparin plasma storage conditions and serum for MPO measurements overmeasures MPO levels.

### EXAMPLE 2

### Centrifugation Studies

Samples were collected from six healthy donors to evaluate the effect of sample handling on MPO values. Both plastic lithium heparin plasma (4 mL, BD 367884) tubes and lithium heparin plasma separator tubes (PST) (4.5 mL, BD 367962) from Becton, Dickinson and Company (BD) were evaluated for the effect of centrifugation speed, clotting time and storage temperature.

The first study evaluated the effect of centrifugation speed and duration. Samples were immediately placed at 2-8 degrees Celsius after completion of collection from each donor and then centrifuged as shown in Table 2:

TABLE 2

| Tube | | |
|---|---|---|
| Number | Time | Speed |
| 1 | 5 min | 1000 × g |
| 2 | 10 min | 500 × g |
| 3 | 5 min | 500 × g |
| 4 | 10 min | 1250 × g |

Samples were then tested immediately with the Architect assay used in Example 1. MPO concentrations were not impacted by centrifugation time or speed when samples are placed at 2-8 degrees Celsius, centrifuged and immediately assayed for MPO. Lithium heparin plasma tubes and PST tubes provided equivalent results.

The next study evaluated the effect of temperature on storage of the sample prior to centrifugation. Upon sample collection tubes were placed at 2-8 degrees Celsius until samples from all donors were collected. Then tubes were placed either at room temperature or at 2-8 degrees Celsius prior to centrifugation. Samples were then centrifuged after 30 minutes, 2 hours or 6 hours. Centrifugation was for 10 minutes at 1250 g. Samples were then tested immediately with the Architect assay used in Example 1. The results are shown in Table 3, including the mean MPO values of the 6 donors at each timepoint and storage condition. Samples in lithium heparin plasma and PST tubes were stable for 6 hours at 2-8 degrees Celsius; however samples in tubes stored at room temperature were unstable and demonstrated a marked increase in myeloperoxidase over time. These data show the potential for significant change in MPO levels due to room temperature storage when a leukocyte MPO secretion inhibitor is not used.

TABLE 3

| Storage | | Li Heparin | Li Heparin PST |
|---|---|---|---|
| Condition | Time | (pmol/L) | (pmol/L) |
| | Baseline | 137 | 141 |
| 2-8 degrees C. | 0.5 hrs | 122 | 111 |
| | 2 hrs | 125 | 96 |
| | 6 hrs | 140 | 116 |
| Room Temp | 0.5 hrs | 152 | 140 |
| | 2 hrs | 286 | 332 |
| | 6 hrs | 913 | 1480 |

### EXAMPLE 3

### Investigation of Additional Tube Types for Sample Stability

Samples were collected from 5 healthy donors to further examine the effect of collection tube type and handling on MPO determinations. Lithium heparin (4 mL, BD 367884), K₂EDTA (4 mL, BD367862), K₂EDTA plasma separator tube (PPT) (5 mL, BD362788) and sodium citrate (2.7 mL, BD363083) collection tubes were purchased from Becton Dickinson and Company. After collection, samples were stored at either room temperature (15-30 degrees Celsius) or at (2-8 degrees Celsius), centrifuged at 0, 2, and 8 hours (10 minutes, 1250 g), plasma removed from the cells and tested using the Architect assay of Example 1 for MPO. EDTA plasma, in either regular EDTA or PPT tubes, was the most stable at both room temperature and 2-8 degrees C. The results shown in Table 4 are the mean MPO values of the 5 donors at each timepoint and storage condition. Citrate and lithium heparin storage tubes were able to maintain MPO levels relatively stable with storage at 2-8 degrees Celsius for 2 hours, note however the MPO concentration in citrate was similar to the concentration measured in EDTA, whereas the concentration in lithium heparin was increased over the concentration in EDTA or citrate at baseline. Storage at room temperature for both citrate and lithium heparin showed marked increase in MPO levels over time. In contrast, use of the storage tubes with the leukocyte MPO secretion inhibitor EDTA was able to maintain MPO levels essentially constant at both room temperature and 2-8 degrees Celsius.

TABLE 4

| Storage | | EDTA | EDTA PPT | Li Heparin | Citrate |
|---|---|---|---|---|---|
| Condition | Time | (pmol/L) | (pmol/L) | (pmol/L) | (pmol/L) |
| | Baseline | 91 | 87 | 130 | 83 |
| 2-8 | 2 hrs | 86 | 88 | 112 | 95 |
| degrees C. | 8 hrs | 99 | 99 | 159 | 100 |
| Room | 2 hrs | 102 | 93 | 465 | 432 |
| Temp | 8 hrs | 112 | 103 | 992 | 1615 |

### EXAMPLE 4

### Stability of Separated Plasma Samples

Samples from 10 normal donors were collected in Lithium Heparin PST (8 mL, BD367964), SST (8.5 mL, BD367988) and EDTA PPT (5 mL, BD362788) tube types. PST and PPT tubes were centrifuged immediately; SST tubes were allowed to clot for 30 minutes at room temperature prior to centrifugation. The isolated plasma or serum were tested neat and spiked with MPO. Samples were then placed at room temperature and tested at time 0, 3.5 hours, 24 hours and 48 hours. Samples were also placed at 2-8 degrees C. and tested using the Architect assay of Example 1 at time 0, 24 hours, 48 hours, 5 days and 8 days. As seen in Tables 5 and 6 the isolated plasma or serum was stable under all conditions tested.

TABLE 5
Room Temperature Stability

| | | Percent Change | | |
|---|---|---|---|---|
| | | from Time 0 | | |
| Tube Type | N | 3.5 hr | 24 hr | 48 hr |
| PST | 13 | −2.5 | −2.9 | −1.7 |
| SST | 10 | −1.3 | −3.6 | −6.5 |
| PPT | 10 | 0.1 | −0.6 | −0.1 |

TABLE 6
2-8 deg. C. Stability

Percent Change
from Time 0

| Tube Type | N | Day 1 | Day 2 | Day 5 | Day 8 |
|---|---|---|---|---|---|
| PST | 13 | −3.9 | −2.7 | −3.3 | −7.0 |
| SST | 10 | −2.4 | −2.1 | −2.5 | −6.8 |
| PPT | 10 | 0.4 | 1.4 | 0.1 | −3.2 |

### EXAMPLE 5

### Spike Recovery Studies

To further investigate the effect of anticoagulant on MPO detection, spike recovery experiments were performed to determine if the presence of anticoagulant in the plasma affected the determination of MPO. Twenty-five matched sets were purchased from BioCollections. Samples were collected into lithium heparin, K₂EDTA and sodium citrate tubes, centrifuged and shipped at 2-8 degrees C. Each sample was >5 mL. Each sample was split into three aliquots. One set was spiked with 300 pmol/L native MPO antigen (Athens Research and Technology, Athens, Ga.) and a second set spiked with 3000 pmol/L MPO antigen. The neat and spiked samples were run on the Architect assay of Example 1 and the Grand Mean percent Difference calculated for each tube type. As seen in Table 7, there was no effect by either the EDTA or citrate storage tubes on MPO recovery; lithium heparin showed a small effect.

TABLE 7

| | % Recovery | % Recovery | |
|---|---|---|---|
| | 300 pmol/L | 3000 pmol/L | % Grand Mean |
| Tube Type | spike | spike | Recovery |
| Heparin | 88 | 94 | 91 |
| EDTA | 102 | 96 | 99 |
| Citrate | 99 | 96 | 98 |

### EXAMPLE 6

### Testing Naturally Elevated Samples in Various Tube Types

Fifty matched specimens were collected from patients presenting to the Emergency Department with chest pain and/or other complaints leading to clinically indicated blood draws. The collection tubes evaluated were plastic serum (SST), K₂EDTA plasma, lithium heparin plasma (PST) and citrate tubes. All samples were stored at room temperature for no more than 60 minutes prior to centrifugation except the EDTA samples that were stored at 2-8 degrees C. as whole blood samples for 24 hours prior to centrifugation. Separated serum and plasma were stored at 2-8 degrees C. until tested by the Architect MPO assay of Example 1. The mean, median, minimum and maximum concentration for each tube type are summarized in the Table 8:

TABLE 8

| | | Mean | Median | Minimum | Maximum |
|---|---|---|---|---|---|
| Tube Type | N | (pmol/L) | (pmol/L) | (pmol/L) | (pmol/L) |
| Serum | 50 | 917.7 | 643.5 | 188.0 | 4882.0 |
| EDTA | 50 | 313.6 | 149.0 | 31.0 | 3791.0 |
| Heparin | 50 | 840.6 | 493.0 | 122.0 | 8252.0 |
| Citrate | 50 | 300.7 | 127.8 | 44.4 | 3501.1 |

These results compare favorably with the results from Example 1. The lowest values are found with EDTA and citrate samples. For most of the samples, the serum sample provided the highest MPO values.

The above-described exemplary embodiments are intended to be illustrative in all respects, rather than restrictive, of the present invention. Thus, the present invention is capable of implementation in many variations and modifications that can be derived from the description herein by a person skilled in the art. All such variations and modifications are considered to be within the scope and spirit of the present invention as defined by the following claims.

What is claimed is:

1. A method for determining concentration of myeloperoxidase (MPO) in a human blood sample comprising:
(a) providing a human peripheral blood sample stored under MPO preservation conditions; and
(b) determining concentration of MPO in a plasma sample derived from the human peripheral blood sample.

2. The method of claim 1 wherein the MPO preservation conditions comprise storage of the human peripheral blood sample in a plasma collection tube containing a leukocyte MPO secretion inhibitor.

3. The method of claim 2 wherein the leukocyte MPO secretion inhibitor comprises a salt of ethylene diamine tetraacetic acid.

4. The method of claim 2 wherein the leukocyte MPO secretion inhibitor comprises a salt of citrate.

5. The method of claim 1 wherein the determining step (b) is performed by a method selected from the group comprising a competitive immunoassay, a sandwich hybridization immunoassay, an enzyme-linked immunosorbent assay, an enzymatic assay or a clinical chemistry assay.

6. The method of claim 2 wherein the determining step (b) is performed with an immunoassay that comprises a sandwich immunoassay using at least one pair of binding antibodies for MPO.

7. The method of claim 1 wherein the MPO preservation conditions comprise maintaining the human peripheral blood sample at a temperature less than 8 degrees centigrade until the plasma sample is derived from the blood sample.

8. The method of claim 7 wherein the determining step (b) is performed by immunoassay.

9. The method of claim 8 wherein the immunoassay comprises a sandwich immunoassay using at least one pair of binding antibodies for MPO.

10. The method of claim 2 wherein the human peripheral blood sample is in a plastic collection container.

11. The method of claim 2 wherein the human peripheral blood sample is in a plastic collection container and the leukocyte MPO secretion inhibitor comprises a salt of ethylene diamine tetraacetic acid.

12. The method of claim 1 wherein the MPO preservation conditions comprise storing the human peripheral blood sample in a plastic container comprising a salt of ethylene diamine tetraacetic acid and maintaining the human peripheral blood sample at a temperature in the range of about two to about eight degrees centigrade for a period of up to about 8 hours before processing of the human peripheral blood sample to produce the plasma sample.

13. The method of claim 1 wherein the plasma sample used in the determining step (b) is derived from the human peripheral blood sample by centrifugation after storage of the human peripheral blood sample at either room temperature or at about two to about eight degrees centigrade for a period of less than about eight hours.

14. The method of claim 1 wherein the MPO preservation conditions comprise storing the human peripheral blood sample in a plastic container comprising a salt of citrate and maintaining the human peripheral blood sample at a temperature in the range of about two to about eight degrees centigrade for a period of up to about 8 hours before processing of the human peripheral blood sample to produce the plasma sample.

15. A method for determining concentration of myeloperoxidase (MPO) in a human blood sample comprising:
(a) providing a human peripheral blood sample stored under MPO preservation conditions;
(b) checking the MPO preservation conditions status of the human peripheral blood sample; and
(c) determining concentration of MPO in a plasma sample derived from the human peripheral blood sample.

16. The method of claim 15 wherein checking the MPO preservation conditions comprises assessment of presence of lysis of leukocytes in the human blood sample.

17. The method of claim 15 wherein checking the MPO preservation conditions comprises assessment of temperature under which the human peripheral blood has been stored.

18. The method of claim 15 wherein the determining step (b) is performed by immunoassay on an automated instrument.

19. The method of claim 18 wherein the immunoassay comprises a sandwich immunoassay using at least one pair of binding antibodies for MPO.

20. The method of claim 18 wherein the determining step (b) is performed by immunoassay on an automated instrument, only after the MPO preservation conditions status is determined to be less than 8 hours of storage at either room temperature or at two to eight degrees Centigrade before processing of the human peripheral blood sample to produce the plasma sample.
